(19) United States
(12) Patent Application Publication
(10) Pub. No.: US 2022/0251549 A1
(43) Pub. Date: Aug. 11, 2022

(54) METHOD FOR CONSTRUCTING CAPTURE LIBRARY AND KIT

(71) Applicant: BERRY GENOMICS CO., LTD, Beijing (CN)

(72) Inventors: Dan Gui, Beijing (CN); Di Chen, Beijing (CN); Guangyuan Wang, Beijing (CN); Jianguang Zhang, Beijing (CN)

(21) Appl. No.: 17/630,022

(22) PCT Filed: Jul. 24, 2020
(86) PCT No.: PCT/CN2020/104351

§ 371 Date: Mar. 25, 2022

(30) Foreign Application Priority Data
Jul. 25, 2019 (CN) 201910678822.8

(51) Int. Cl. C12N 15/10

(52) U.S. Cl. CPC C12N 15/1093

(57) ABSTRACT

The present invention provides a method for constructing a capture library, comprising the steps of: (1) obtaining fragmented DNAs; (2) connecting the fragmented DNAs with a Y-shaped linker to obtain a pre-library; (3) hybridizing the pre-library and a hybridization probe in the absence of a blocking sequence to obtain hybridization products; and (4) performing a PCR amplification on the hybridization products to obtain the capture library. The present invention also provides to a kit for carrying out the method.

### TECHNICAL FIELD

The present invention belongs to the field of molecular biology, specifically relates to a method for constructing a hybrid capture library and a kit.

### BACKGROUND

Exon capture is a technique that uses a probe to capture and enrich the DNA sequences of exon region, which is widely used in scientific research and clinical detection. Compared with whole genome sequencing, it has a lower cost, a shorter cycle, a better coverage, and is more economic and efficient. The construction of traditional exon capture library generally includes the following steps: genomic DNA fragmentation, end-repair and end-addition of A, followed by ligation of a linker and a tag sequence, obtaining pre-library by a first round of PCR amplification, hybridizing the pre-library with a hybridization probe in the presence of a blocking sequence, and purification followed by a second PCR amplification to obtain a final capture library (see FIG. 1). The linker and tag sequence are of great significance for on-board sequencing, sample discrimination and tracing source of the original DNA molecule.

However, during the first round of PCR amplification to construct pre-library, the linker and tag sequence tend to form longer (around 60 bp at each end), reverse complementary sequence structures. Such sequence structures may readily anneal to each other during hybridization capture such that non-target sequences are captured together when the probe binds to the specific sequences, thereby reducing the overall capture specificity. Therefore, the non-target sequences other than the inserted sequences need to be effectively blocked during hybridization capture in case that non-specific binding occurs. Presently, the blocking sequence employs sequence that is reverse complementary to the linker sequence, and the blocking of the linker is accomplished by base-complementary pairing with the linker sequence. Specifically, the blocking sequence is divided into two parts, one part is reverse complementary to sequence of amplification primer P5 and sequencing primer 1 (also referred to as Read 1 sequencing primer) and the other one is reverse complementary to sequence of sequencing primer 2 (also referred to as Read 2 sequencing primer), index tag and amplification primer P7, and linker blocking is performed by complementary pairing with its counterpart. However, the binding of such linker blocking sequences tends to be affected by temperature during hybridization, and a dimer is readily formed between the blocking sequences, resulting in a reduced blocking efficiency and a further reduced capture efficiency of target region. In addition, in high-throughput sequencing, typically, a large number of samples are involved, and multiple tag sequences are required for discrimination. The above-mentioned blocking strategy means that the blocking sequence needs to be designed separately for each tag sequence, which undoubtedly increases the complexity of experimental operation and cost of library construction.

In order to control the cost, strategies have been proposed to block the tag sequence with corresponding number of hypoxanthines, i.e., to modify the end of tag sequence with hypoxanthines instead of adding the additional blocking sequence. However, hypoxanthine has a certain preference for blocked bases, resulting in a poor blocking effect on some tag sequences, thus affecting the capture efficiency. Meanwhile, synthesis of hypoxanthine is expensive. A bridge blocking design strategy has also been proposed, i.e., corresponding blocking sequences are designed for linker sequences at both ends of target fragments, respectively, and a bridge connection using 6-8 C3 arms is adopted for tag sequence located in the middle part. CN108456713A also proposes blocking modification of linker end, such as reverse dT modification, interarm modification, amino modification, and ddNTP modification, thereby achieving the blocking of the linker sequence. However, either strategy requires addition of additional blocking sequence or special blocking modification to linker with a limited assistance in controlling hybridization cost.

In addition, to increase the diversity of sequencing library and ensure the library abundance, it is generally required that the amount of DNAs (i.e., the amount of pre-library) used for hybridization capture is 500 ng or higher. For example, the kits commonly used in hybridization capture, twist Human Core Exome EF Singleplex Complete Kit, 96 Samples (Twist Bioscience, Cat No. 100790) and xGen® Exome Research Panel v1.0 (IDT, Cat No. 1056115), both require the initial amount of at least 500 ng of pre-library for hybridization, whereas SureSelect^{XT} HS Target Enrichment System for Illumina Paired-End Multiplexed Sequencing Library (Agilent Technologies, Cat No. G9704N) requires the initial amount of 500-1000 ng of pre-library for hybridization. Due to requirement of DNA amount for pre-library and loss of DNA for purification step, the traditional capture library construction method requires a PCR amplification to amplify the amount of DNAs extracted from genome and to compensate for the above loss due to purification, so as to meet the requirements of the hybridization capture reaction by providing a sufficient amount of pre-library. Therefore, a simple and economical method of constructing capture library is in need, which can effectively reduce non-specific binding during hybridization and improve capture efficiency.

### SUMMARY

In view of the above problems in construction of capture library, in order to save cost and simplify the tediousness in library construction process, the inventors have proposed a method for constructing a capture library without a PCR pre-amplification for pre-library, wherein the method does not require addition of blocking sequence or end modification to the linker.

The present invention is based on the following two facts discovered by the inventors: (1) at an initial amount of 5-50 ng DNAs, a good coverage and a coverage uniformity can also be achieved in the obtained pre-library without a PCR amplification. Therefore, a large amount of pre-library (500 ng-1000 ng) is not essential for hybridization capture, and a PCR pre-amplification is not a necessary step for constructing the pre-library; (2) by connecting the fragmented DNAs to a Y-shaped linker, a blocking sequence used to block the linker and tag sequence can be omitted from hybridization capture without any impact on the capture efficiency, coverage and uniformity of coverage, thereby saving the hybridization capture cost.

Accordingly, in the first aspect, the present invention provides a method of constructing a capture library comprising the following steps:

(1) obtaining fragmented DNAs;

(2) connecting the fragmented DNAs with a Y-shaped linker to obtain a pre-library;

(3) hybridizing the pre-library with a hybridization probe in the absence of a blocking sequence to obtain a hybridization product;

(4) performing a PCR amplification on the hybridization product to obtain the capture library.

In one embodiment, the fragmented DNAs refer to natural short-fragment DNAs or short-fragment DNAs obtained by artificial disruption of genomic DNAs. In one embodiment, the fragmented DNAs are derived from blood, serum, plasma, joint fluid, semen, urine, sweat, saliva, stool, cerebrospinal fluid, ascites, pleural fluid, bile, pancreatic fluid, and the like. In a preferred embodiment, the natural short-fragment DNAs are peripheral blood free DNAs, tumor free DNAs or naturally degraded genomic DNAs. In another embodiment, the genomic DNAs may be of a variety of origins, e.g., peripheral blood, dried blood spot, buccal swab, and the like. The person skilled in the art is aware of a method for disrupting genomic DNAs, e.g., by a sonication, a mechanical disruption or an enzymatic digestion, and the like. Since the sonication and mechanical disruption lose relatively much DNAs, it is preferable for DNA fragmentation with the enzymatic digestion in the presence of a little initial amount of DNAs (e.g., as low as 50 ng).

In one embodiment, the fragmented DNAs are 150-400 bp in length, preferably 180-230 bp.

In one embodiment, the method of the invention further comprises the steps of end repair and/or end-addition of A of the fragmented DNAs prior to being ligated to the Y-shaped linker (i.e., step 2). In this embodiment, the DNAs can be end-repaired using any enzyme known to those skilled in the art suitable for end-repair, such as T4 DNA polymerase, Klenow enzyme, and mixture thereof. In this embodiment, the DNAs can be end-added with A using any suitable enzyme for end-addition of A known to those skilled in the art. Examples of such enzymes include, but not limited to, Taq enzyme, klenow ex-enzyme, and mixture thereof. In this embodiment, end repair and end-addition of A may be carried out in two reaction systems, i.e., end-addition of A may be performed after end-repair followed by purification. Alternatively, and preferably, the steps of end-repair and end-addition of A are performed in one reaction system, i.e., end-repair and end-addition of A are made simultaneously, followed by purification of the nucleic acid. Alternatively, and more preferably, the steps of DNA fragmentation, end repair, and end-addition of A are performed in one reaction prior to ligation of the linker. This not only simplifies the procedure and saves cost, but also reduces contamination between samples.

In one embodiment, the incubation time and temperature used for end-filling and end-addition of A can be determined by those skilled in the art according to routine technique in line with specific demand.

In one embodiment, step (2) may be performed with any enzyme suitable for the ligation of the linker known to those skilled in the art. Examples of such enzymes include, but not limited to, T4 DNA ligase, T7 DNA ligase, or mixtures thereof. Conditions for carrying out the ligation reaction are well known to those skilled in the art.

In the context of the present invention, a “Y-shaped linker” refers to a linker formed by two strands that are not completely complementary, wherein one end of the linker forms a duplex due to complementarity between bases of the two strands, and the other end does not form a duplex due to incomplete complementarity between bases of the two strands. Currently commonly used Y-shaped linker mainly includes a long Y-shaped linker (FIG. 3a) and a truncated Y-shaped linker (FIG. 3b). As shown in FIG. 3a, a conventional long Y-shaped linker mainly comprises amplification primer sequence (P5/P7), index tag sequence, read 1/read 2 sequencing primer sequence and index read sequencing primer sequence, wherein the sequences of read 1/read 2 sequencing primer sequence and index read sequencing primer are not completely complementary to form a partial double-strand. As shown in FIG. 3b, a conventional truncated Y-shaped linker mainly comprises read 1/read 2 sequencing primer sequence and index sequencing primer sequence, or partial read 1/read 2 sequencing primer sequence and partial index sequencing primer sequence, wherein the sequences of the read 1/read 2 sequencing primer and index read sequencing primer are not completely complementary to form a partial double strand. Such truncated Y-shaped linker generally needs to be used in conjunction with an additional linker comprising P5/P7 primer and an index tag sequence.

For example, the Y-shaped linker available in the present invention comprises sequences of two strands as follows:

SEQ ID NO: 1
5'-AATGATACGGCGACCACCGAGATCTACACTCTTTCCCTAC
ACGACGCTCTTCCGATCT-3'
SEQ ID NO: 2
(with phosphorylation modification at 5' end)
5'-GATCGGAAGAGCACACGTCTGAACTCCAGTCAC{index }
ATCTCGTATGCCGTCTTCTGCTTG-3'

Wherein, the complementary portions of two strands are underlined.

Methods for phosphorylation modification of oligonucleotide are well known to those skilled in the art. For example, oligonucleotide can be phosphorylated at 5′ end by polynucleotide kinases, or phosphate group can be added directly to 5′ end when primer is synthesized.

In one embodiment, step (3) of the method of the present invention is carried out in a liquid phase hybridization system.

In the context of the present invention, a “blocking sequence” refers to a sequence used to block linker and tag sequence, including a sequence designed to be complementary to linker and/or tag sequence. In some embodiments, to increase the blocking effect of the blocking sequence, a specific modification is conducted at ends of the blocking sequence, such as a reversed dT modification, an amino modification, a ddNTP modification (including ddCTP, ddATP, ddGTP, and ddTTP), a spacer modification, a hypoxanthine modification, a random base modification, and the like.

In a traditional capture library construction, a PCR amplification is typically performed after ligation of linker and tag sequence to amplify the amount of target DNAs, thus ensuring the efficiency of subsequent hybridization steps and meeting the requirements of on-board sequencing. In order to reduce specific binding and increase target penetration, it is often necessary to add the blocking sequence to hybridization system that function to block the amplified linker and tag sequence by base complementarity so that they do not interfere with the binding of target sequence to hybridization probe during hybridization. However, since the blocking sequence is base-complementary to linker and tag sequence, it can not only bind to linker and tag sequence, but also to each other during hybridization. Such binding between the blocking sequences may result in unsatisfactory blocking, thereby reducing capture efficiency. Furthermore, given that multiple tag sequences (sometimes up to 96) are required for simultaneous sequencing of multiple samples, it is required to design the blocking sequence separately for each tag sequence, increasing the difficulty of subsequent sequencing data analysis and experimental cost.

Unexpectedly, the inventors found that a better capture efficiency can be achieved in the case of using the Y-shaped linker without PCR pre-amplification for preparation of pre-library in hybridization system without addition of any blocking sequence.

Thus, in one embodiment, a system for hybridization includes a hybridization buffer, Cot-1 DNAs, and a hybridization probe, but no blocking sequence. The conditions for hybridization, such as hybridization temperature, hybridization time and the like, can be adjusted by one skilled in the art according to actual demand. The general principle for designing and preparing hybridization probe is also well known to those skilled in the art.

Method for performing step (3) PCR amplification

In a second aspect, the invention provides a kit for constructing a capture library comprising:

(1) reagents for connecting a linker, including a Y-shaped linker;

(2) reagents for hybridization, excluding a blocking sequence;

(3) reagents for a PCR amplification.

In one embodiment, the reagents for hybridization include a hybridization buffer, Cot-1 DNAs, and a hybridization probe, but no blocking sequence.

In one embodiment, the reagents for PCR amplification include buffer, PCR polymerase and amplification primer.

In one embodiment, the capture library prepared according to the method of the invention may be used on various Next-generation sequencing platforms, including but not limited to sequencing platforms such as Roche/454 FLX, Illumina/Hiseq, Miseq, NextSeq, and Life Technologies/SOLID system, PGM, proton, and the like.

The excellent technical effects of the present invention lie in: (1) the requirement for the initial DNA amount is relatively low, even as low as 5 ng, which greatly improves the utilization ratio of rare samples and expands the application range of the present invention. For example, the method and kit of the present invention can be applied to the sample types with dry blood spot, buccal swab, cfDNA and the like, which are not suitable for common exon capture process due to a small extraction amount of DNAs; (2) the library construction process is simple, and the method of the present invention does not need a PCR reaction before obtaining pre-library, and thus the pre-library construction can be completed in only about 2 hours, while the construction of pre-library in conventional capture library construction method takes about 6 hours; (3) since the method of the present invention does not include a blocking sequence in hybridization system, substantial saving in library building cost can be achieved while ensuring that capture efficiency and coverage are unaffected.

The invention will now be described in more detail by way of examples with the accompanying drawings. It should be understood by those skilled in the art that the drawings and their examples are for illustrative purposes only and are not to be construed as limiting the invention in any way. The embodiments and features of the embodiments in the present application can be combined with each other without contradiction.

### BRIEF DESCRIPTION OF THE DRAWINGS

FIG. 1: a scheme of a conventional capture library construction method.

FIG. 2: a scheme of one embodiment of the capture library construction method of the present invention.

FIGS. 3a and 3b: a schematic diagram of the Y-shaped linker structure.

FIG. 4: a schematic diagram of the blocking sequence structure.

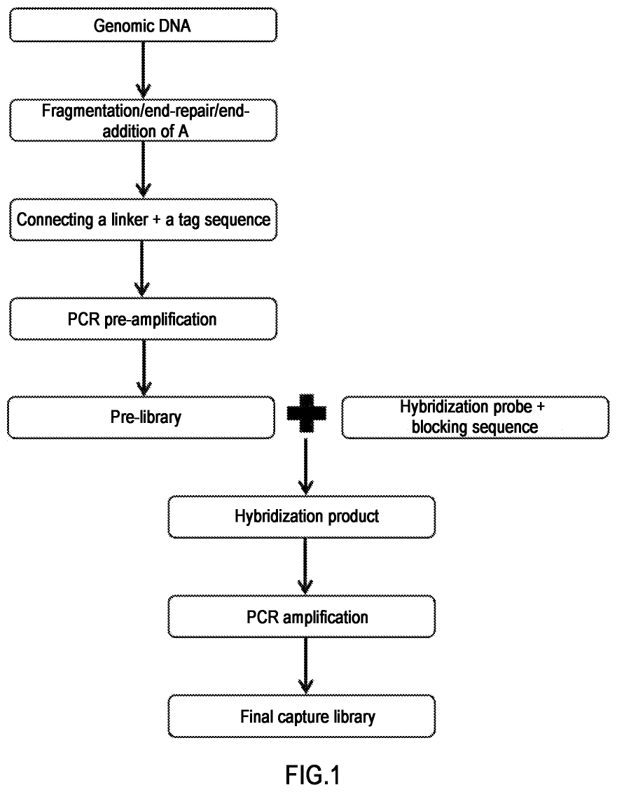

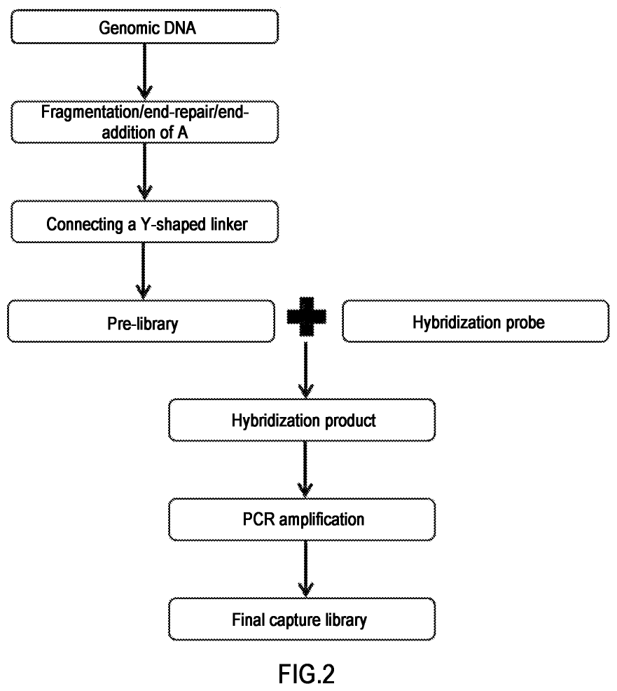

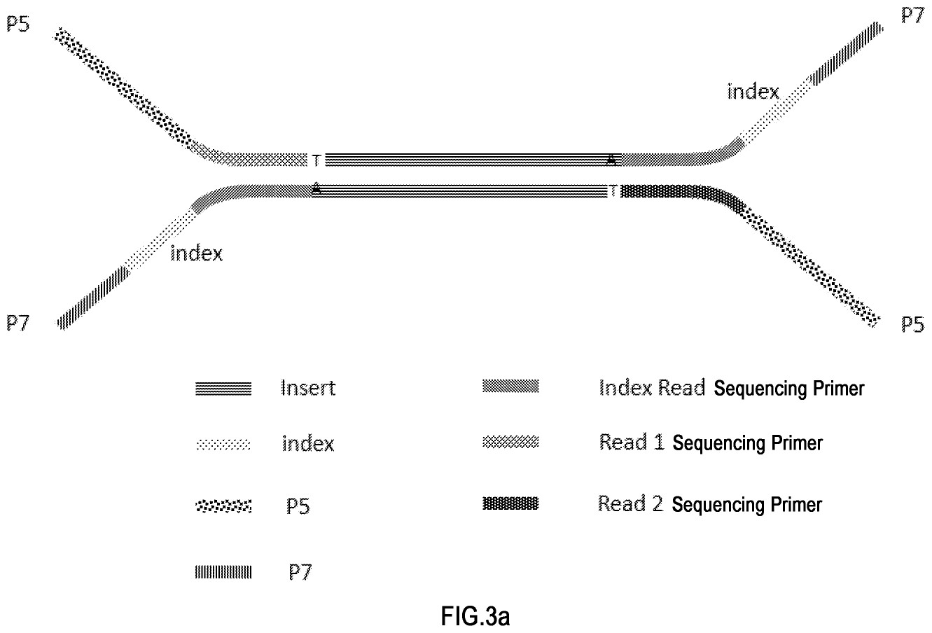

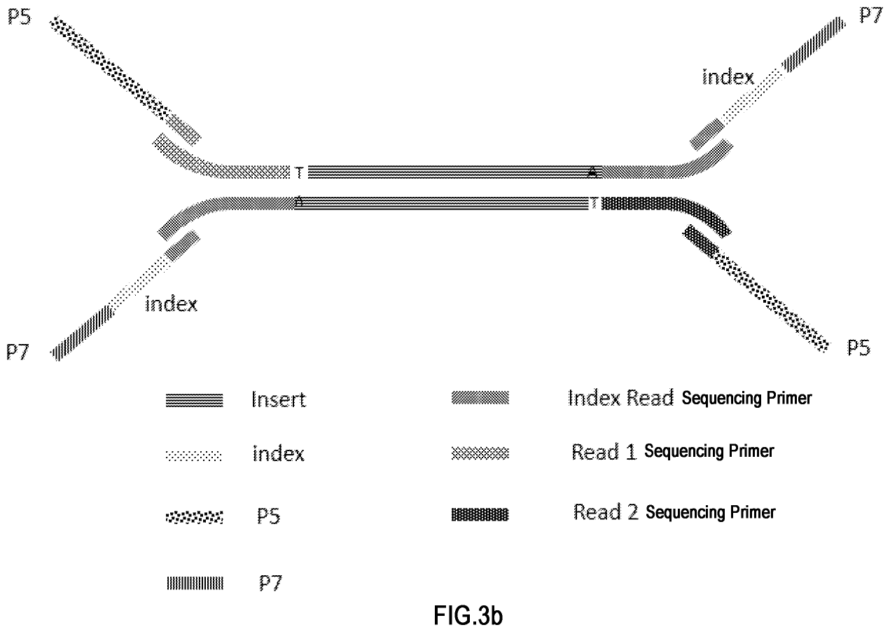

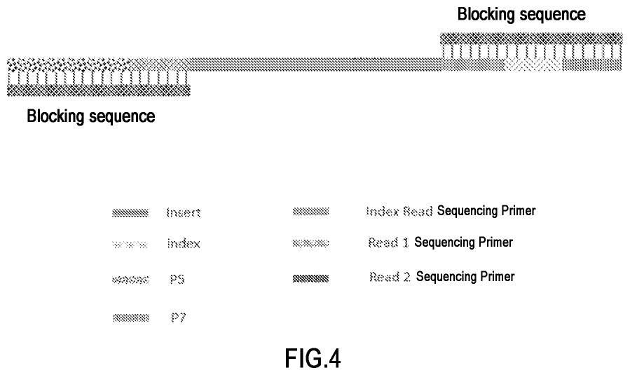

### DETAILED DESCRIPTION

### Example 1: Constructing a Capture Library According to the Method of the Present Invention

Step 1. Obtaining Fragmented DNAs, End Repair and End-Addition of A

According to the manufacturer's instructions, the reaction system shown in Table 1 was prepared with 5×WGS Fragmentation Mix kit (Enzymatics, Cat No. Y9410L) to complete the fragmentation, end-repair and end-addition of A in one step and reacted according to the following procedure: 4° C., 1 min; 32° C., 16 min; 65° C., 30 min and then held at 4° C.

TABLE 1

| Genomic DNAs | 50 | ng |
|---|---|---|
| 10 × Fragmentation Buffer | 2.5 | μl |
| 5 × WGS Fragmentation Mix | 5 | μl |
| Enzyme-free water | up to 25 μl | |
| Total volume | 25 | μl |

Step 2. Connecting a Linker

(1) Preparation of the Linker

Sequences shown as SEQ ID NO: 1 and SEQ ID NO: 2 were synthesized with phosphorylation modification at 5′ end of SEQ ID NO: 2.

SEQ ID NO: 1
5'-AATGATACGGCGACCACCGAGATCTACACTCTTTCCCTA
CACGACGCTCTTCCGATCT-3'
SEQ ID NO: 2
5'-GATCGGAAGAGCACACGTCTGAACTCCAGTCAC{index}
ATCTCGTATGCCGTCTTCTGCTTG-3'

The sequences shown as SEQ ID NO: 1 and SEQ ID NO: 2 were annealed under the following procedure to form a long Y-shaped linker: 95° C., 2 min; 95° C., 2 min, cooled to 90° C. at rate of 0.1° C./s for 2 min; cooled to 85° C. at rate of 0.1° C./s for 2 min; cooled to 80° C. at rate of 0.1° C./s for 2 min; and so on, until cooled to 25° C. at rate of 0.1° C./s for 2 min; finally held at 4° C.

(2) Ligation of the Linker

Using WGS Ligase Kit (Enzymatics, Cat No. L6030-WL), ligation system as shown in Table 2 was prepared with the reaction system of step 1 and incubated at 20° C. for 15 minutes and then held at 4° C.

TABLE 2

| | | |
|---|---|---|
| Reaction system of Step 1 | 25 | μl |
| 5X Ligation Buffer | 10 | μl |
| Y-shaped linker | 5 | μl |
| T4 ligase | 5 | μl |
| Enzyme-free water | 5 | μl |
| Total volume | 50 | μl |

After ligation, the ligation product was purified using the Beckman Agencourt AMPure XP Kit (Beckman, Cat No. A63882).

Step 3: Capturing Hybridization

Using xGen Lockdown Reagents Kit (IDT, Cat No. 1072281), 14.5 μl of hybridization reagent (9.5 μl xGen 2×hybridization buffer, 3 μl xGen hybridization buffer enhancer and 2 μl Cot-1 DNAs) was added to the purified product of step 2, mixed thoroughly, and incubated for 10 minutes at room temperature. After the incubation, 12.75 μl of the supernatant was added to a new low adsorption 0.2 mL centrifuge tube, followed by addition of 4.25 μl hybridization probe. At the end of incubation, immediately after sufficient mixing, the following program was run: 95° C. 30 s; 65° C., 1 min, 37° C., 3 s, 60 cycles; 65° C. 16 hours; then kept at 65° C.

After hybridization, the hybridization product (i.e., magnetic beads binding to the target sequence) was washed and purified with xGen Lockdown Reagents Kit (IDT, Cat No. 1072281) according to the manufacturer's instructions.

Step 4: PCR Amplification

The amplification system shown in Table 3 was prepared with 2×KAPA HiFiHotStartReadyMix Kit (KAPA, Cat No. KK2602) according to the manufacturer's instructions and PCR was performed according to the following procedure: 95° C. 45 s; 98° C. 15 s, 65° C. 30 s, 72° C. 30 s, 12 cycles; 72° C. 1 min; then held at 4° C.

Sequences of the amplification primers are as follows:

(SEQ ID NO: 3)
P5 primer: 5'-AATGATACGGCGACCACCGA-3';
(SEQ ID NO: 4)
P7 primer: 5'-CAAGCAGAAGACGGCATACGA-3'.

TABLE 3

| | | |
|---|---|---|
| DNAs (w/beads) | 23 | μl |
| 2 × KAPA HiFiHotStartReadyMix | 25 | μl |
| 25 μM P5 + P7 primer mix | 2 | μl |
| Total volume | 50 | μl |

After completion of PCR program, the product was purified using Beckman Agencourt AMPure XP Kit (Beckman, Cat No. A63882) to obtain the final capture library.

### Comparative Example 1

The library construction method of the example was substantially same as that of Example 1, except that 2 μl blocking sequence was further included in hybridization reagent of step 3, wherein the blocking sequence was xGen Universal Blockers—TS Mix (IDT, Cat No. 1075475).

### Comparative Example 2

The library construction method of the example was same as that of Example 1, except that after step 2, the purified product was subjected to a PCR pre-amplification to prepare a pre-library, and 2 μl of blocking sequence was added to hybridization reagent of step 3. Specifically, the pre-amplification system as shown in Table 4 was prepared with 2×KAPA HiFiHotStartReadyMix Kit (KAPA, Cat No. KK2602) and PCR was performed according to the following procedure: 95° C. 45 s; 98° C. 15 s, 65° C. 30 s, 72° C. 30 s, 7 cycles; 72° C. 1 min; then held at 4° C.

Sequences of the pre-amplification primers are as follows:

(SEQ ID NO: 3)
P5 primer: 5'-AATGATACGGCGACCACCGA-3';
(SEQ ID NO: 4)
P7 primer: 5'-CAAGCAGAAGACGGCATACGA-3'.

TABLE 4

| | | |
|---|---|---|
| Purified product of step 2 | 23 | μl |
| 2 × KAPA HiFiHotStartReadyMix | 25 | μl |
| 25 μM P5 + P7 primer mix | 2 | μl |
| Total volume | 50 | μl |

After completion of PCR program, the product was purified using Beckman Agencourt AMPure XP Kit (Beckman, Cat No. A63882) followed by capture hybridization. The blocking sequence added to the hybridization reagent in step 3 was xGen Universal Blockers—TS Mix (IDT, Cat No. 1075475).

### Comparative Example 3

The library construction method of this example was same as that of Comparative Example 2, except that in Step 3, no blocking sequence was added to hybridization system.

The capture libraries prepared in Example 1 and Comparative Examples 1-3 above were subjected to a qPCR quantification, and then sequenced (150 bp double-ended sequencing) using Illumina NovaSeq 6000 sequencing platform according to the standard protocol of sequencer, with 10 G of data measured for each sample. The sequencing result is shown in Table 5.

TABLE 5

| | Capture | | | Alignment |
|---|---|---|---|---|
| | efficiency | 4x coverage | 20x coverage | ratio |
| Example 1 (without a PCR pre- | 67.89% | 99.28% | 98.46% | 91.93% |
| amplification, without a blocking | | | | |
| sequence) | | | | |
| Comparative Example 1 (without | 64.97% | 99.32% | 98.83% | 92.55% |
| a PCR pre-amplification, with a | | | | |
| blocking sequence) | | | | |
| Comparative Example 2 (with a | 62.93% | 99.47% | 98.79% | 92.44% |
| PCR pre-amplification, with a | | | | |
| blocking sequence) | | | | |
| Comparative Example 3 (with a | 26.53% | 99.22% | 88.54% | 92.44% |
| PCR pre-amplification, without a | | | | |
| blocking sequence) | | | | |

As can be seen from Table 5, in the absence of the PCR pre-amplification, there's no significant effect on the final capture efficiency, coverage and alignment ratio with or without addition of the blocking sequence, and the capture libraries prepared all meet the quality requirements for sequencing (Example 1 vs. Comparative Example 1).

Furthermore, after comparing the sequencing result of Comparative Example 1 with Comparative Example 2, it is found that there's no significant difference in the capture efficiency, coverage, and alignment ratio in the case of addition of the blocking sequence, indicating that the PCR preamplification can be omitted without affecting the quality of the final library. However, after comparing the sequencing result of Example 1 with that of Comparative Example 3, it is found that without addition of the blocking sequence, the PCR pre-amplification results in a significant decrease in quality control parameters such as capture efficiency and 20×coverage.

Finally, comparing Comparative Examples 2 and 3, it can be seen that, in the case of the PCR pre-amplification, the absence of addition of the blocking sequence results in a significant decrease in quality control parameters such as capture efficiency and 20×coverage. This indicates when the pre-library is formed by DNAs connected to the linker with the PCR pre-amplification, the blocking sequence must be added during hybridization with hybridization probe, otherwise the quality of final capture library would be seriously affected, resulting it unable to meet the requirements of on-board sequencing and subsequent data analysis.

### Example 2: Constructing a Capture Library According to the Method of the Present Invention

According to the method described in Example 1, capture libraries were prepared using peripheral blood gDNAs, dried blood spot gDNAs, and buccal swab gDNAs, respectively. The capture library was quantified by qPCR, and then sequenced using Illumina NovaSeq 6000 sequencing platform according to the standard sequencer operating procedure (150 bp double-ended sequencing), and 10 G data was measured for each sample. The sequencing result is shown in Table 6.

TABLE 6

| | Capture | 4x | 20x | Alignment | Repetition |
|---|---|---|---|---|---|
| Sample Type | efficiency | coverage | coverage | ratio | ratio |
| Peripheral | 65.91% | 99.28% | 98.73% | 92.64% | 17.89% |
| blood gDNAs | | | | | |
| Dried blood | 67.56% | 99.33% | 98.73% | 91.25% | 17.98% |
| spot gDNAs | | | | | |
| Buccal swab | 65.08% | 99.29% | 98.75% | 91.32% | 14.58% |
| gDNAs | | | | | |

As can be seen from table above, the construction method of the sequencing library of the present invention is applicable to a variety of sample types, especially samples such as peripheral blood, dried blood spot, buccal swab, and the like with a low content of DNAs.

### Example 3: Effect of Initial Amount of DNAs on Capture Library

Capture libraries were constructed using different starting amounts of genomic DNA samples according to the method described in Example 1. The capture library was quantified by qPCR, and then sequenced using Illumina NovaSeq 6000 sequencing platform according to the standard sequencer operating procedure (150 bp double-ended sequencing), and 10 G data was measured for each sample. The sequencing result is shown in Table 7.

TABLE 7

| Initial | | Capture | | | Alignment |
|---|---|---|---|---|---|
| amount | | efficiency | 4x coverage | 20x coverage | ratio |
| 5 | ng | 66.04% | 99.22% | 97.46% | 91.50% |
| 10 | ng | 66.70% | 99.28% | 98.60% | 91.55% |
| 20 | ng | 64.56% | 99.24% | 98.72% | 92.12% |
| 30 | ng | 64.18% | 99.25% | 98.72% | 92.00% |
| 40 | ng | 64.57% | 99.23% | 98.77% | 92.04% |
| 50 | ng | 67.19% | 99.16% | 98.61% | 92.00% |
| 80 | ng | 67.16% | 99.23% | 98.63% | 91.39% |
| 100 | ng | 66.99% | 99.27% | 98.80% | 91.50% |
| 200 | ng | 64.34% | 99.27% | 98.74% | 91.98% |

As can be seen from the table above, the capture libraries constructed according to the method of the present invention do not differ from each other significantly in the capture efficiency, coverage, and alignment ratio in range of 5 ng to 200 ng. This indicates that the method according to the present invention can be used with a sample having an initial DNA amount as low as 5 ng and that the capture library prepared fully meets the requirements for on-board sequencing and subsequent data analysis.

It should be noted that the above-mentioned embodiments illustrate preferably rather than limit the invention, and those skilled in the art will be able to design many alternative and various embodiments. It will be understood by those skilled in the art that various changes, equivalent replacement, and improvement may be made therein within the protection of the invention without departing from the spirit and scope of the invention.

1. A method of constructing a capture library comprising the steps of:
(1) obtaining fragmented DNAs;
(2) connecting the fragmented DNAs with a Y-shaped linker to obtain a pre-library;
(3) hybridizing the pre-library and a hybridization probe in the absence of a blocking sequence to obtain a hybridization product;
(4) performing a PCR amplification on the hybrid product to obtain the capture library.

2. The method of claim 1, wherein the fragmented DNAs are natural short-fragment DNAs or short-fragment DNAs obtained by artificial disruption of genomic DNAs.

3. The method of claim 2, wherein the natural short-fragment DNAs are peripheral blood free DNAs, tumor free DNAs or naturally degraded genomic DNAs.

4. The method of claim 2, wherein the artificial disruption of the genomic DNAs is made by a sonication, a mechanical disruption, or an enzymatic digestion.

5. The method of claim 1, wherein the fragmented DNAs are derived from blood, serum, plasma, joint fluid, semen, urine, sweat, saliva, stool, cerebrospinal fluid, ascites, pleural fluid, bile, or pancreatic fluid.

6. The method of claim 1, wherein the fragmented DNAs are 150-400 bp in length.

7. The method of claim 6, wherein the fragmented DNAs are 180-230 bp in length.

8. The method of claim 1, further comprising the step of end repair and/or end-addition of A to the fragmented DNAs prior to step (2).

9. The method of claim 8, wherein the steps of end repair and end-addition of A are performed in one reaction system.

10. The method of claim 8, wherein the steps of DNA fragmentation, end repair, and end-addition of A are performed in one reaction system.

11. The method of claim 1, wherein the Y-shaped linker is a long Y-shaped linker or a truncated Y-shaped linker.

12. The method of claim 11, wherein the long Y-shaped linker comprises amplification primer, index tag sequence, read 1/read 2 sequencing primer, and index read sequencing primer.

13. The method of claim 11, wherein the truncated Y-shaped linker comprises read 1/read 2 sequencing primer and index sequencing primer, or partial read 1/read 2 sequencing primer and partial index sequencing primer.

14. The method of claim 1, wherein the blocking sequence comprises sequences designed to be reverse complementary to the linker and/or the tag sequence.

15. The method of claim 1, wherein step (3) is carried out in a liquid phase system.

16. A kit for constructing a capture library comprising:
(1) reagents for connecting a linker, including a Y-shaped linker;
(2) reagents for hybridization, excluding a blocking sequence;
(3) reagents for a PCR amplification.

17. The kit of claim 16, wherein the Y-shaped linker is a long Y-shaped linker or a truncated Y-shaped linker.

18. The kit of claim 17, wherein the long Y-shaped linker comprises amplification primer sequence, index tag sequence, read 1/read 2 sequencing primer sequence, and index read sequencing primer sequence.

19. The kit of claim 17, wherein the truncated Y-shaped linker comprises read 1/read 2 sequencing primer sequence and index sequencing primer sequence, or partial read 1/read 2 sequencing primer sequence and partial index sequencing primer sequence.

20. The kit of claim 16, further comprising reagents for performing end repair and/or end-addition of A.

21. The kit of claim 16, wherein the reagents for hybridization comprises a hybridization buffer, Cot-1 DNAs, and a hybridization probe, while does not include a blocking sequence.

22. The kit of claim 16, wherein the blocking sequence comprises sequences designed to be reverse complementary to the linker and/or the tag sequence.

23. The kit of claim 16, wherein the reagent for PCR amplification comprises a buffer, a PCR polymerase and an amplification primer.

24. A capture library constructed according to claim 1, wherein the capture library is used for next-generation sequencing platform.


SEQUENCE LISTING (4 sequences)

SEQ ID NO: 1 - DNA, 58 bp, artificial sequence
  Description of Artificial Sequence Synthetic oligonucleotide
aatgatacggcgaccaccgagatctacactctttccctacacgacgctcttccgatct

SEQ ID NO: 2 - DNA, 57 bp, artificial sequence
  Description of Artificial Sequence Synthetic oligonucleotide
gatcggaagagcacacgtctgaactccagtcacatctcgtatgccgtcttctgcttg

SEQ ID NO: 3 - DNA, 20 bp, artificial sequence
  Description of Artificial Sequence Synthetic oligonucleotide
aatgatacggcgaccaccga

SEQ ID NO: 4 - DNA, 21 bp, artificial sequence
  Description of Artificial Sequence Synthetic oligonucleotide
caagcagaagacggcatacga